(19) United States
(12) Patent Application Publication
(10) Pub. No.: US 2004/0097990 A1
(43) Pub. Date: May 20, 2004

(54) Graft material, device & method of making

(76) Inventor: Iris Ginron Zhao, Philadelphia, PA (US)

Correspondence Address: Dr. Iris G. Zhao, 1925 Chestnut Street, # C-20, Philadelphia, PA, 19103, US

(21) Appl. No.: 10/600,364

(22) Filed: Jun. 21, 2003

Related U.S. Application Data
(63) Continuation-in-part of application No. 09/589,248, filed on Jun. 7, 2000.
Application No. 09/589,248 is a continuation-in-part of application No. 09/240,832, filed on Jan. 27, 1999, now Pat. No. 6,171,635.

(51) Int. Cl. A61B 17/08

(52) U.S. Cl. 606/153

(57) ABSTRACT

This novel graft system comprises a material, device, and method of making. The material is a solidable adhesive fluid, suitable to form an extravascular solid bond. The device is selected from ice, laser, balloon, and needle. The method is to making an openings on a vessel wall. Blood flow is used to induce vessel cells to spread out from the opening so that a graft is produced in situ. The best embodiment is a reversed bypass from an artery to a vein network. This system is also useful for repairing tubular gland, ureter, fallopian tube, and lymphduct.

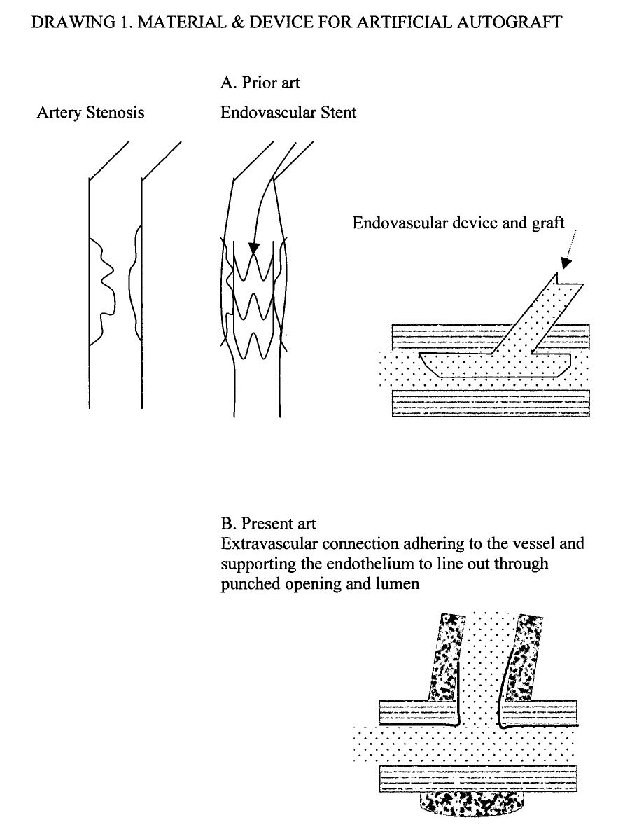

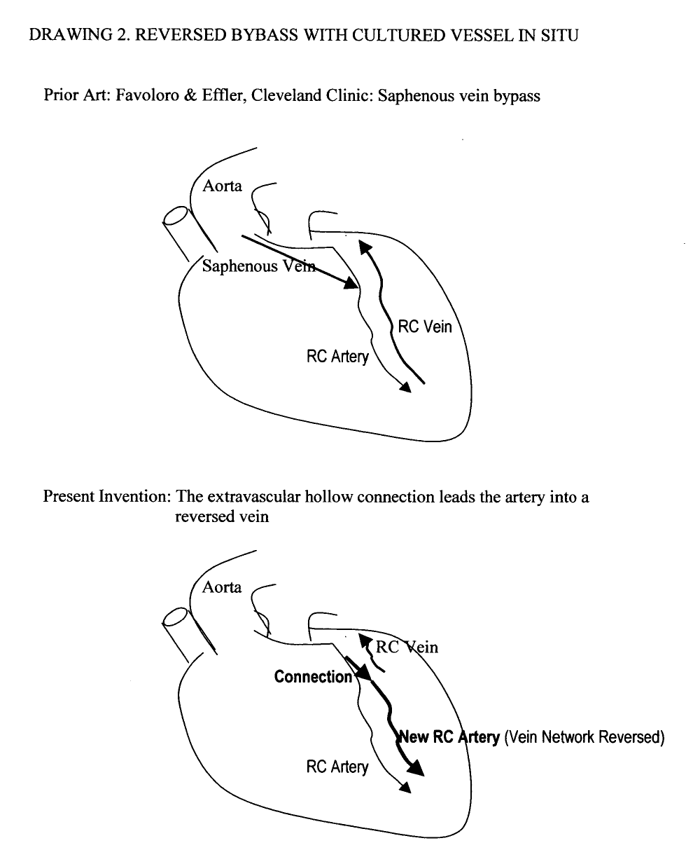

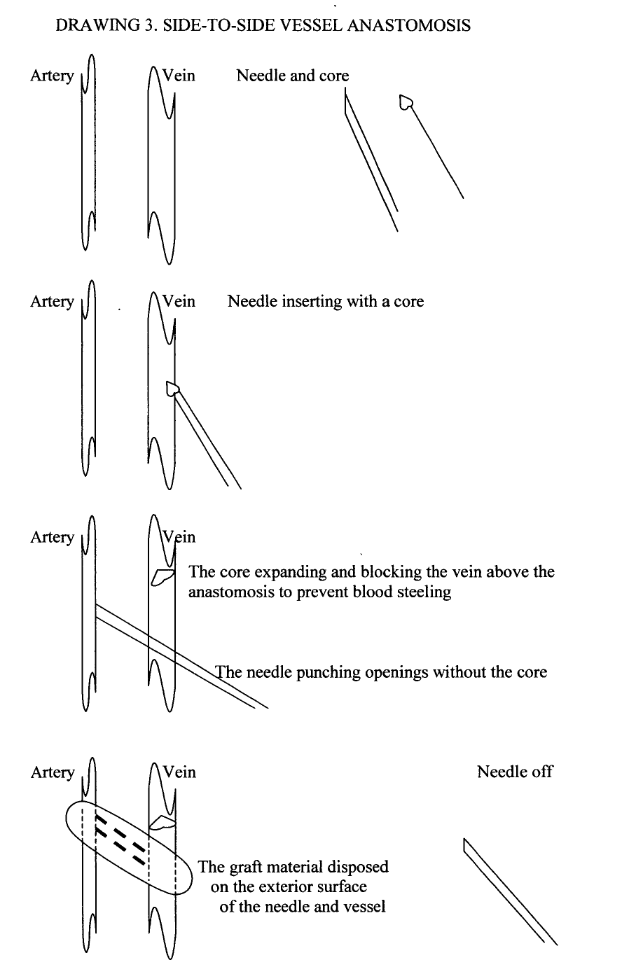

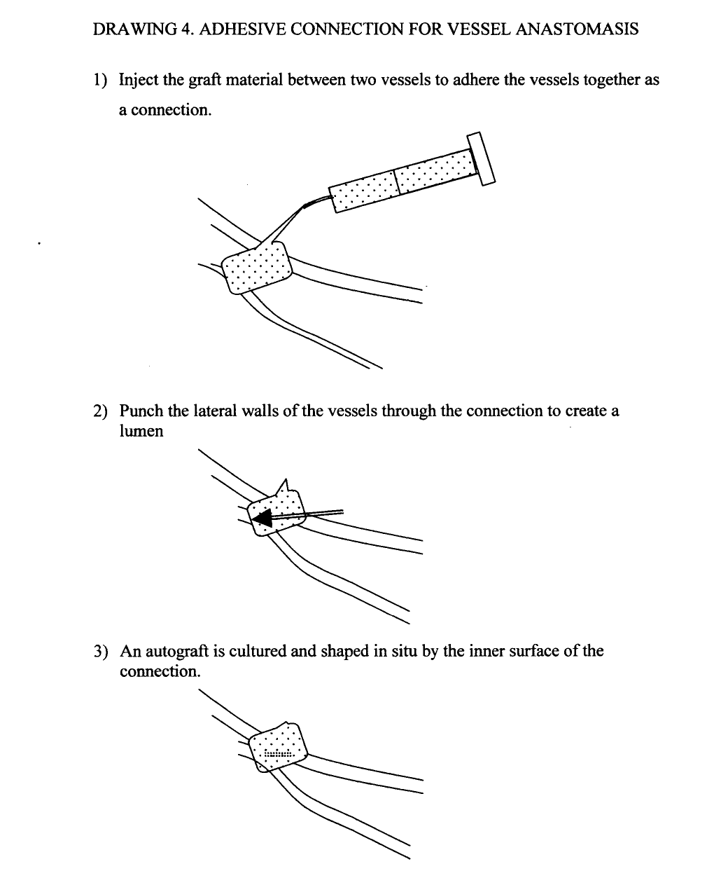

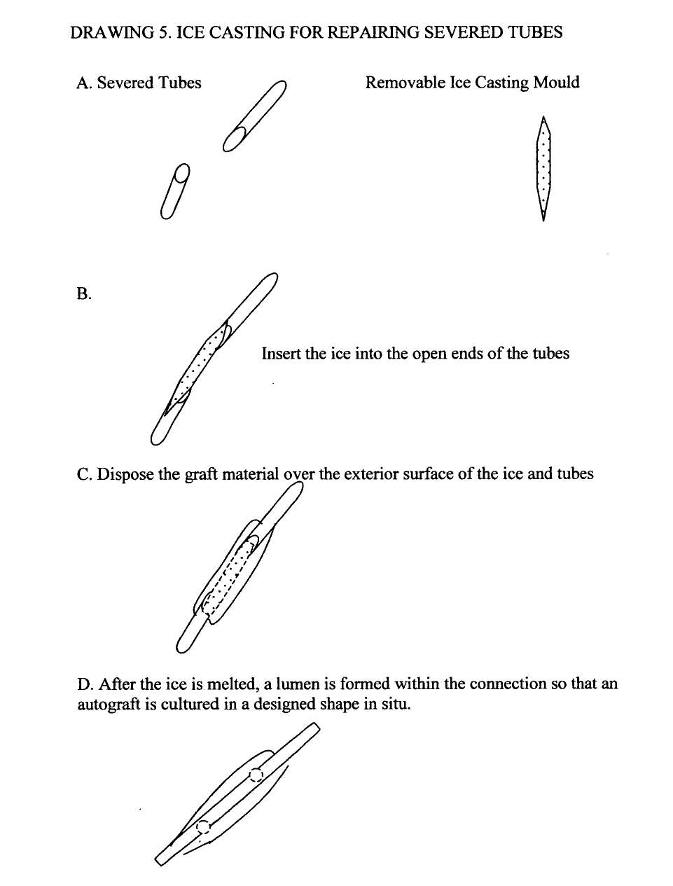

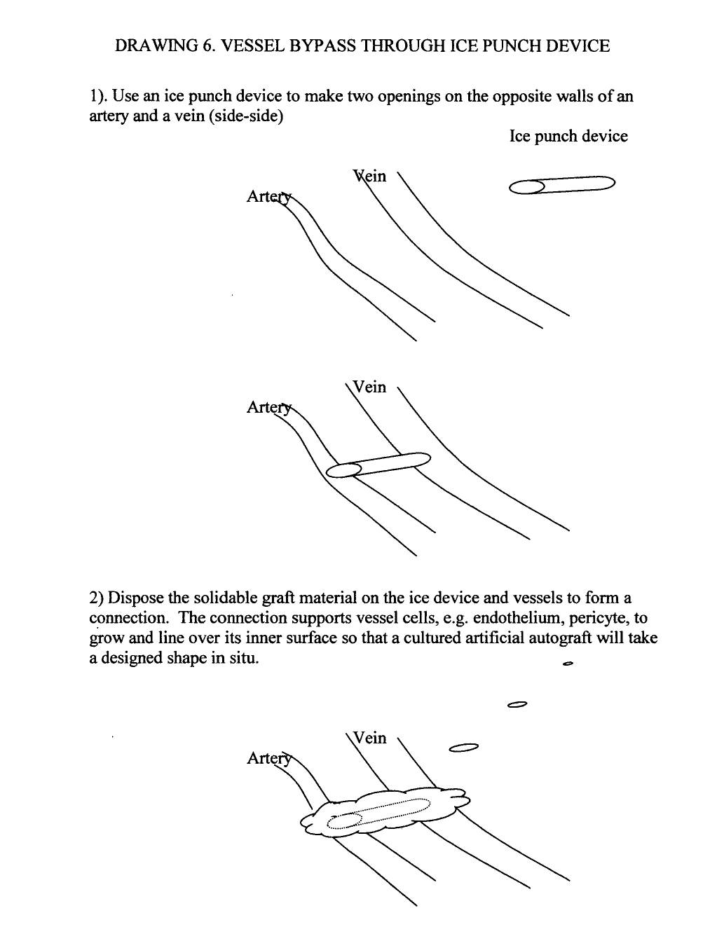

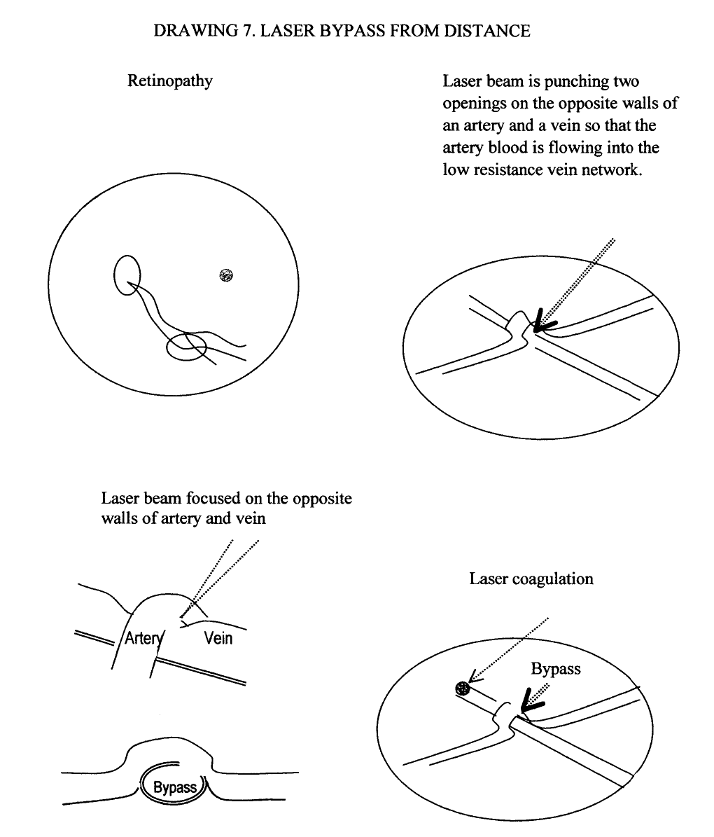

### CROSS REFERENCES TO RELATED APPLICATIONS

This application is a continuation-in-part of application Ser. No. 09/589,248, filed Jun. 7, 2000, now U.S. Pat. No. 6,______, which is a continuation-in-part of application Ser. No. 09/240,832, filed Jul. 20, 1998, now U.S. Pat. No. 6,164,281.

### BACKGROUND OF THE INVENTION

1. Technical Field

The present invention relates to an artificial vessel graft system including the material, device, and method of making.

2. Description of the Related Art

Each year, over 600,000 coronary artery bypass surgery are performed worldwide. Various inventions have been proposed to help the body circulation. For example, U.S. patent issued to Miyata et al. U.S. Pat. No. 4,098,571 for heterograft; to Chanda et al U.S. Pat. No. 5,645,587 for preventing calcification and degeneration of implanted grafts; to Katsuen et al U.S. Pat. No. 5,691,203 for serum-free culture of human vascular endothelia cells, to Edelman et al U.S. Pat. No. 5,766,584 for inhibiting vascular smooth muscle cell proliferation with implanted matrix containing vascular endothelial cells; to Epstein et al U.S. Pat. No. 5,951,589 for expansile device used in blood vessels; to Krajicek U.S. Pat. No. 5,968,089 for internal shield of a anastomosis; to Rateliff et al U.S. Pat. No. 5,968,090 for a endovascular graft and method; to Kranz U.S. Pat. No. 5,968,093 for a stent comprising at least one thin walled, tubular member. However, high distal resistance speeds up atherosclerosis. Consequently, bypass surgery may have to be done repeatedly.

Besides, it is difficult to sew vessels having a caliber (lumen diameter) smaller than 0.2-1 mm. Autografts are not always available. Hetergrafts can cause rejection. Thus, it is desirable to culture an autograft product in situ. This novel technology breaks the lower limitation of vessel caliber requirement. Plus, the invention gets dividing blood flow to reality. The smaller the vessel is, the less pressure and structure difference exists between the artery and vein, and the better the outcome can be.

### SUMMARY OF THE INVENTION

It is a primary object of the present invention to provide a convenient artificial graft system. This system includes a graft material, device and method of making.

A further object of this invention is to provide a reversed bypass from an artery to a vein network through a cultured vessel in situ.

A still further object of this invention is to provide a novel extravascular connection for vessel anastomosis.

### BRIEF DESCRIPTION OF THE DRAWINGS

Drawing. 1 shows the differences between prior arts and the present system.

Drawing. 2 shows a reversed bypass from an artery to an adjacent vein network.

Drawing. 3 is an embodiment of the graft material and device for vessel anastomosis.

Drawing. 4. is an embodiment, showing the graft material gluing vessels.

Drawing. 5. is an embodiment, showing an ice device for repairing severed tubes.

Drawing. 6. is an embodiment, showing an ice punch device for vessel bypass.

Drawing. 7. is an embodiment, showing a laser punch for retina vessel bypass.

### DETAILED DESCRIPTION OF THE INVENTION

1. Concept of the Invention

Upon discovering the dynamic force of blood flow leading neovascularization, the present invention is using blood flow to culture new vessels in situ. Previously, if a person is 70 years old, all other existing vessels should be 70 years old. Presently, the new vessels should not at the same living expectation as the old ones.

Because vein and its network are usually spared from artery stenosis, thus, this artery→vein network (A→V) bypass reduces stenosis. In addition, to reduce hyperdisplasia, A→V aneurysm, and obstruction consequences following prior art, the invention may divide only 20-70% by volume of blood flow from an artery into adjacent vein network.

Main blood pressure and vessel resistance are raised by small artery (lumen 50μ-1 mm), arterioles (20-50μ) and precapillary sphincter at metarteriole (10-15μ). In the fingers and palms, short channels connect arterioles with venules and bypass capillaries. These arteriovenous (A-V) anastomosis and shunts have thick, muscular walls and abundant innervations, presumably by vasoconstrictor nerve fibers (William G. Ganong, Review of Medical Physiology, p 550-553, 1999). Although the application connects a small artery to a small vein also, it acts to increase capillary blood flow rather than to steal blood from lower reaches of supplied area. The vein under the A-V connection is opened to an artery so that the blood in the artery will flow through increased cross-sectional channels. The vein above the connection is closed so that the blood return above it is not disturbed. The method makes A and V from “ [symbol] ” to “ [symbol] ”. Sewing is not necessary and thus, the limitation for sewing is overcome. This volume reduction reduces at least 20% resistance. Since the total cross sectional area of venule is close to the sectional area of capillaries that is 10 times bigger than small arteries and arterioles, activating 10% by volume reserved venule system, by theory, would cover all small artery supplied area. The new growing vessel is supposed to be innervated by the nerve arising from the budding edge of artery. Thus, the new vessel inherits the characteristics from parent artery. Thus, the risk of recurrent stenosis is greatly reduced and consequently the long-term success is increased.

U.S. Pat. No. 6,164,281 indicates how a vessel hemorrhage turns into a new vessel and the uses thereupon. Since neovascularization is the late stage of severe hypoxemia, micoaneurysm is actually an attempt to get more blood supply. Red blood cells come from higher pressure end of an arteriole and exist on its lower pressure end. If the neck is too small for the two, single red blood cell will come and leave one by one in order to keep the circuit. The body is struggling so hard for every red blood cell to come through. The applicant disagrees with traditional photocoagulation. Particularly, treatments shall not suppress such struggle and seal the neck of the circuit. Rather than coagulating the neck of a microaneurysm, the application activates unused vein compensative network to improve the blood supply. Since neovascularization has a growth peak in about 3-7^{th} day after a surgery, the release of vessel growth enhancers is designed to stop as soon as the new vessel is formed.

Microaneurysms are formed when a bleeding whirls in loose tissue stria until exhausted at the center of the whirl. Under the condition of hypoxemia or stress, a blood stream may not clot. Even a groove of tissue stria may reside a blood flow well within a living body. When a blood stream flows within a groove, channel or supporting tissue, vascular endothelial cells are very likely to grow and reach another blood stream successfully. When bleeding constantly striking on a vessel wall, that wall will open and accept it.

2. Autograft Materials and Connection (Drawings 1-7)

MATERIAL The present invention provides an adhesive nonpyogenic fluid suitable to form to a solid in a designed shape. The basic matrix can be the body component of the same person who will receive the body component, e.g., own blood. Drawing some blood is more convenience than cutting a piece of vessel from the body. Generally, bleeding becomes a solid clot attached to the wounded vessel. Blood coagulation takes about 2-8 minutes. The graft material can be absorbed and leave the growing cover cells as a cultured autograft in situ.

This graft material must be nonpyogenic. It has two phases:

1) a first fluid phase to be positioned (disposed) surrounding a body fluid and joined to the adjacent tissue of such body fluid, and

2) above first fluid phase turning into a second solid-like phase to support and seal the body fluid.

Fibrin, trunk cell, embryo stem cell, umbilical cell, pericyte, endothelium, epithelium, connective tissue component, clone, and their combination can be added to enhance a desirable neovascularization.

Heparin can keep the blood flowing more vividly. Sodium citrate is a natural composition in the body. Coral eye prosthesis was the first material allowing new vessels to grow. Alginate works well on a watery surface. Other option is selected from surgical acceptable adhesives, e.g., fibrin, collagen, alginate, sugar, silicone, acrylic, hyaluronate, healon, and polyethylene. Trypsin and collagenase help endothelium spread.

More adding is selected from: anti-coagulation agent, endothelium adhesion agent, endothelium and epithelium growth factor, vessel dilating agent, angiogenesis factor, heparin and analogue, viagra and analogue, adhesive peptide, adenosine, arginine, alanine, arginine, asparagines, serine, tyrosine, glycine, glutamic acid, valine, isoleucine, cyclohexyl, butyloxycarbonyl, chitosan, fibroblast growth factor, transforming growth factors α and β, tissue factor V, vitreous body component, angiogenin, platelet derived endothelial cell growth factor, angiogenic herb extract, circulation enhancer, transferrin, laminin, fibronectin, or vitronectin. The solid-like phase may further contain gas microbubbles, e.g. oxygen. The advantages of containing O₂ bubble are increasing the elasticity and the growth of new vessel (neovascularization).

This graft material is viscous glue-like liquid. It will form a solid in about 0.3 second to 30 minutes. The solidation of the material is accelerated by contacting air, rough surface, moisture, ultraviolet light (cross-link), or temperature change. The blood from the patient self can be mixed with adhesives, e.g., fibrin. This system is not only useful for bypass and vessel anastomosis, but also for repairing vasculature and tubular organs, e.g., ureter, fallopian tube, and lymphoduct.

Connection (Drawings 1-7)

DRAWING 1. B This solidable adhesive nonpyogenic material is shaped into a novel extravascular connection, e.g., coating, cuff, and wrapping. This connection works like a casting mould, lumen wall, and position bond. The connection is adhered (joined) to the adjacent tissue of a vessel opening. The lumen of the connection is aligned with the openings of old vessels. The wall of the connection joins firmly to the exterior surface of the old vessel wall. The smooth interior surface of the connection is extended from the interior surface (endothelium or epithelium) of a natural vessel. Thus, the endothelial cells from the cutting edge can expand out as a bud. The newly formed vessel inherits characteristics from the original artery as its distal branch.

Differed from prior endovascular graft, this connection means an extravascular bond. The connection contains: 1) a body fluid, 2) a lumen, and 3) an extra vascular coating joined securely to the old vessel or tubular organ. The connection may even contain a hook to secure its position. The connection takes any designed shape. It is very simple and cost save. The graft glue holds and prevents the end of the artery contracting or shrinking back. Once the vessel autograft is formed, the most part of the extravascular support may have been absorbed. Saphenous vein, xenografts, sutures, clips, and stents are not necessary.

DRAWING 4. The invention is useful for vessel anastomosis. Glue can reduce the distance between vessels and hold their relative position like “><”. For example, 1) glue and press the exterior surface of two vessels together to ensure a firm bond; 2) punch two openings on the opposite walls of these two vessels through the glue to form a joint opening and lumen communicating these two vessels; 3) the graft glue become a bypass paving connection having a wall and a lumen. The new lumen has a smooth interior surface for endothelium to expand and line over. Preferably, this adhesive extravascular connection will be absorbed and thus, these two vessels can go back close to its original position. Hopefully, when the distance is increased, the endothelium growth will follow through.

3. Devices for Making Artificial Autograft (Drawings 3-7)

The graft device is removable and will not occupy spaces. The graft material is positioned on the exterior surface of this removable device to form a solid coating so that after removing such device, the lumen created by the device is supported by such solid coating. The feature of the device is making a lumen within a solid for a body fluid to flow. The solid means vessel wall, solidable graft material, tissue, organ, and their combination. The device is selected from needle, laser, ice, water soluble pharmaceutical solid, e.g., EDTA or derivatives, body composition, e.g., high density lipid (HDL), balloon, and their combination. Devices made of water-soluble body compositions, e.g., sodium citrate, is unlikely to block a blood flow. Laser beams do not stay. The ice-casting probes and ice punch devices can be easily melted and absorbed. The water-soluble solid are body fluid compositions. Oxygen exists in the body.

According to general human vessel data, the wall thickness of 30μ lumen diameter arteries is about 20μ and the wall thickness of 30μ lumen diameter veins is only 3μ. It is desirable for the microsurgery punch device to create a small orifice, hole or flap in a range or equivalence about 10μ-3 mm caliber diameter in plane and 2μ-1.5 mm in depth. Approximately 20-100% by volume of blood flow in a donor artery is leading into a receiving vein, or distal artery through above opening.

More samples are illuminated in Drawing 3—Punch needle with a core, Drawing 4—Glue application, Drawing 5—Ice casting device, and Drawing 6—Ice punch device.

The ice is make of a material selected from water; saline; body fluid imitation, e.g., BSS by Alcon Inc.; blood substitute; transfusion solution; pharmaceutical solution, e.g., NaCitrate, heparin; biobeneficial agent, and their mixture. It has a solid phase under 0-4° C. and a fluid phase above 0-4° C.

The term of “punch” means to create an opening on a solid comprising vessel walls, organ, tissue, solidable adhesive material, and their combination. The term of “pass” means only going through. For example, the needle of Drawing 3 is passing the first wall of a receiving vessel with a core, and thereafter punching two openings on the second wall of said receiving vessel and the first wall of a donor vessel to form a joint opening on the opposite walls of the two vessels.

ENERGY SOURCES Since the focused area and allowable punch depth on vessel walls are calculated by micron (μ), special efforts must be made to reach required resolution and sharpness. The energy of laser photon should be for freeing gaps and junctions rather than burning the surrounding, which reduces reduce neovascularization. Current “ArF excimer laser” is for refractive surgery. A 193 nm argon excimer laser device may interact with vessel wall in a photochemical evaporation mode.

The shape of the punched opening is a circle or flap like “ [symbol] ”. Cutaneous catheter can be introduced from distance. Chemical resolvent and gap opener is helpful, e.g., trypsin, collagenase, EDTA, heparin, erosive acid, lipolysis agent, and hypertonics.

DRAWING 7. Many arteries have to curl (curve) to cross veins in the retina. Such curling increases the resistance, accelerates atherosclerosis, and reduces the blood flow. When a small artery and vein are sharing same sheath, the connection can just be a blood flow. Laser beams can evaporate a focused tissue to form an opening from a curling artery to its adjacent vein through their opposite walls. Thus, the artery blood flows strait forward to a low-pressure vein network. Joint sheath and increased intraocular pressure reduce bleeding.

The length of the new-grown vessel graft is equal to the length of the lumen. Such lumen and graft can be short or long. The anastomosis can be side to side e.g., A→V (Drawings 2-4, 6, 7), A→A, vessel→organ; or end to end, e.g., ureter→ureter (Drawing 5).

The opening and lumen made by the graft device has a diameter from 20μ to 3 mm. Approximately 20-100% by volume of blood flow in a donor vessel can be divided into a receiving vessel.

4. Method of Make Artificial Autograft (Drawings 1-7)

The concept of the present invention is to make an opening on the wall of a tubular organ. The term of tubular organ means blood vessel, lymph vessel, tubular gland, and/or hollow organs. The opening is held to open by a solidable adhesive material so that the body fluid and cover cells can go through. This solidable adhesive material is joined to the adjacent tissue surrounding the opening to form a smooth interior surface for surface cells (endothelium, epithelium, cover cells) of the tubular organ to spread out and lined over. The new vessel grows out from the vessel opening as a branch extended from the parent. The new branch is a cultured vessel in situ in a designed shape.

The present invention produces artificial branches for natural vessels. The opening edge must be fresh cut. No device or graft should stay inside the lumen because it blocks the paving path. The fluid flowing out from the opening must be supported. The surface from one lumen and opening to another lumen and opening should be smooth united (joined together) so that the endothelium or epithelium can line over on a continuous surface. The surface does not have to be high dense. A loose tissue supports a blood flow well too. Pores small than 7μ in diameter is good enough for red blood cells to flow through. Pericytes is also essential for a healthy artery.

The graft material is positioned (put, disposed) to an exterior surface comprising a fluid, lumen, cavity, vessel, tissue, organ, device, balloon, or their combination to form a solid bond (coating). The coating is to 1) secure the relative position of two tubular organs; 2) support epithelial or endothelial cells to spread and line over; and 3) seal the body fluid.

An embodiment of this application (Drawing 2.) is a reversed bypass for reducing peripheral vessel resistance and increasing blood supply and perfusion to an ischemia tissue, area, or organ in a living mammal, including:

1) selecting a narrow artery that is causing ischemia in a tissue, area, or organ, (“Narrow” means causing ischemia.)

2) selecting an adjacent vein that is carrying blood for the same area and can be spared from vein blood return, multiple vein lateral system, or volume conserve vein system,

3) making an opening and lumen to connect such artery and vein so that 20%-100% by volume blood in the artery is flowing into the vein network through such opening and lumen,

4) blocking the vein above the point of the connection,

5) establishing a pressure gradient in the created new lumen within such connection so that the direction of blood flow in the vein network will be constantly reversed.

Because sleep helps wound recovery, rejuvenation, and normalizing cortisol level, a therapeutic effective amount of an anxiolytic drug or a hypnotic drug can be added. For example, sodium thiopental, chlorpromazine, chloral hydrate, diazepam, clonazepam, essential amino acid, and valium.

Having described the present invention, discovery, and the preferred embodiment, modifications and equivalents of the disclosed concepts may occur to on of ordinary skill in the art. Such equivalents and modifications are considered to be within the scope of the invention and are intended to be embrace by the following claims.

What is claimed and desired to be secured by the United States patent is:

1. An artificial graft for sealing and holding a body fluid within a living mammal comprising an adhesive nonpyogenic fluid suitable to form a solid surrounding and sealing a body fluid.

2. An artificial graft for sealing and holding a body fluid within a living mammal comprising a connection made of a solidable adhesive nonpyogenic material, wherein said connection having a lumen and a wall joined to the lumens and the walls of said two tubular organs respectively.

3. An artificial graft for sealing and holding a body fluid within a living mammal comprising:
i) a first fluid phase surrounding a body fluid and joining to the adjacent tissue of a body fluid, and
ii) said first fluid phase turning into a second solid-like phase to support and seal said body fluid.

4. The solidable adhesive nonpyogenic material of claim 1 is disposed around an opening of a tubular organ to support the interior surface cell of said tubular organ spreading out from said opening.

5. The solidable adhesive nonpyogenic material of claim 1 is disposed on the exterior surface of a removable device and a tubular organ suitable to form a solid bond, wherein after removing said removable device, a lumen is formed within said solid bond.

6. The artificial graft of claim 1 comprising a basic matrix made of a blood component from a mammal who will receive said blood component.

7. The artificial graft of claim 1 comprising fibrin, collagen, trunk cell, stem cell, umbilical cell, pericyte, endothelium, epithelium, embryo, clone, body fluid composition, or a combination thereof.

8. The artificial graft of claim 1 comprising calcium, coral component, alginate, polyethylene, hyaluronate, healon, silicone, acrylic, adhesive peptide, anti-coagulation agent, endothelium adhesion agent, endothelium growth factor, endothelium and epithelium growth hormone, trypsin, vessel dilating agent, collagenase, angiogenesis factor, oxygen microbubble, heparin and analogue, viagra and analogue, adenosine, arginine, alanine, arginine, asparagines, serine, tyrosine, glycine, glutamic acid, valine, isoleucine, cyclohexyl, butyloxycarbonyl, chitosan, sugar, fatty acid, surgical acceptable adhesive, fibroblast growth factor, transforming growth factors α and β, vitreous body component, angiogenin, platelet derived endothelial cell growth factor, angiogenic herb extract, transferrin, laminin, fibronectin, vitronectin, and a combination thereof.

9. The artificial graft system according to claim 1 further comprising a removable device selected from the group consisting of a laser, ice in a designed shape, water-soluble solid in a designed shape, needle, balloon, and a combination thereof.

10. The removable device of claim 9 is a punch device suitable to make an opening within a solid comprising the vessel wall, organ, tissue, solidable nonpyogenic material, and a combination thereof, wherein approximately 20-100% by volume of blood flow in a donor vessel is flowing into a receiving vessel through said opening.

11. The removable device of claim 9 is a laser device.

12. The removable device of claim 9 is a needle passing the first wall of a receiving vessel with a core, and thereafter punching the second wall of said receiving vessel and the first wall of a donor vessel to form a joint opening on the opposite walls of said vessels.

13. The removable device of claim 9 is an ice made of saline, body fluid substitute, blood substitute, transfusion solution, pharmaceutical solution, biobeneficial agent, water, or a mixture thereof.

14. The method of making an artificial graft of claim 1 comprising making an opening on the wall of a tubular organ.

15. The method of making an artificial graft of claim 1 comprising:
connecting two lumens of two tubular organs through a device, wherein said device is coated by a solidable adhesive material joined to the adjacent tissue of said two lumens, and thereafter,
removing the device to leave a lumen that is connecting said two lumens of said two tubular organs.

16. The method of making an artificial graft of claim 1 comprising:
a) selecting an artery and a vein related to same ischemia area,
b) binding said artery and vein together by a solidable adhesive nonpyogenic material,
c) blocking the vein above b), and
d) making an opening and lumen on the opposite walls of said vein and artery through said solidable adhesive nonpyogenic material to allow the cover cells from the edge of the opening spreading out on the surface of said lumen to produce a vessel graft in situ.
